(19) United States
(12) Patent Application Publication
(10) Pub. No.: US 2018/0360917 A1
(43) Pub. Date: Dec. 20, 2018

(54) SINCALIDE FORMULATIONS AND METHODS OF USE

(71) Applicant: Apicore LLC, Somerset, NJ (US)

(72) Inventors: Vijal Kumar, Mendham, NJ (US); Ravishanker Kovi, Monroe, NJ (US); Raghu Rami Reddy Kasu, Monmouth Junction, NJ (US); Venkat Manukondu, Dayton, NJ (US)

(21) Appl. No.: 16/009,945

(22) Filed: Jun. 15, 2018

Related U.S. Application Data
(60) Provisional application No. 62/520,254, filed on Jun. 15, 2017.

(51) Int. Cl. A61K 38/22; A61K 9/00; A61K 47/18; A61K 47/02; A61P 1/16

(52) U.S. Cl. CPC A61K 38/2207; A61K 9/0019; A61P 1/16; A61K 47/02; A61K 47/183

(57) ABSTRACT

The present invention relates to improved Sincalide ready-to-use formulations. The presently disclosed formulations eliminate the need for freeze drying and reconstitution with water for injection at the time of use. The present invention also relates to Sincalide formulations characterized by improved stability and ease of commercial manufacturing.

### FIELD OF THE INVENTION

The invention relates to pharmaceutically acceptable compositions in the form of ready-to-use aqueous pharmaceutical formulation of Sincalide Injection. Further, the present invention discloses process for the preparation of said stable ready-to-use aqueous pharmaceutical preparation.

### BACKGROUND OF THE INVENTION

Sincalide for Injection, USP is a cholecystokinetic drug administered by injection to aid in diagnosing disorders of the gallbladder and pancreas. It is the 8-amino acid C-terminal fragment of cholecystokinin, and also known as CCK-8,

The active pharmaceutical ingredient, 1-De(5-oxo-L-glutamine-5-L-proline)-2-de-L-me-thioninecaerulein or “Sincalide” (CAS# 25126-32-3), is a synthetically prepared C-terminal octapeptide of cholecystokinin (CCK-8), with the following amino acid sequence: Asp-Tyr(S0₃H)-Met-Gly-Trp-Met-Asp-Phe-NH₂.

KINEVAC® (Bracco Diagnostics) was first introduced in 1976 for use in stimulation of gallbladder contraction and pancreatic secretion and/or intestinal motility for diagnostic purposes, and was finished as a sterile, nonpyrogenic, lyophilized white powder in a 5-mL (nominal) glass vial to contain Sincalide with 45 mg sodium chloride to provide tonicity; sodium hydroxide or hydrochloric acid may have been added for adjustment (pH 5.5-6.5). The type I glass vial was sealed under a nitrogen headspace with a Tompkins B0849 closure. This two-ingredient formulation was incorporated into the U.S. Pharmacopeia/National Formulary, USP 24, NF 19, Jan. 1, 2000.

Since its introduction, various drawbacks in the manufacturing and analysis of KINEVAC® have been identified. For example, the two-ingredient formulation suffers from potency variability. This variability was exacerbated by the fact that the formulation was analyzed using a guinea pig gallbladder contraction bioassay for potency of both Sincalide and KINEVAC®. This bioassay was unable to distinguish between bioactivity of Sincalide and bioactivity of Sincalide degradants. Accordingly, a 20% overage of Sincalide was required in previous Sincalide formulations to compensate for the limitations of the bioassay. Thus, there is a need for Sincalide formulations having improved and consistent potency as established by a Sincalide specific assay such as HPLC.

### SUMMARY CF THE INVENTION

The current approved KINEVAC® formulation contains Sincalide as a sterile lyophilized powder. The formulations include a variety of excipients, such as, for example, antioxidants, buffers, bulking agents/tonicity adjusters, chelating agents, complexing agents, crosslinking agents, co-solvents, osmolality adjustors, solubilizers, surfactants, stabilizers, pH adjustors, lyo-protectants/cryo-protectants, air/liquid and/or ice-liquid interface protectants (protectants against surface induced denaturation), freeze-thaw protectants, protectants against protein/peptide denaturation, protectants for rehydration, and wetting agents. The marketed formulations include excipients that perform the functions of at least: (i) a bulking agent/tonicity adjuster, (ii) a stabilizer, (iii) a surfactant, (iv) a chelator, and (v) a buffer. Lyophilized product is stored at room temperature. The lyophilized powder reconstituted with 5 ml water for injection is contained in a vial which contains 1 mcg/ml Sincalide, 170 mg mannitol and other ingredients namely, 30 mg arginine hydrochloride, 15 mg lysine hydrochloride, 9 mg potassium phosphate, dibasic, 4 mg methionine, 0.04 mg sodium metabisulfite, and 0.005 μg polysorbate 20. After reconstitution, the vial can be stored in the refrigerator for use within 12 hours. Normal dosing is 0.02 micrograms per kilogram patient weight. Thus, a 70 kg patient would receive 1.4 mcg or 1.4 ml.

Disclosed herein are improved Sincalide ready-to-use formulations. The presently disclosed formulations eliminate the need for freeze drying and reconstitution with water for injection at the time of use. The Sincalide formulations of the invention are also purer than prior art formulations, and have fewer degradants and more consistent potency. In addition, the purity of these formulations may be assessed by HPLC/LC-MS, thus eliminating the need for the bioassay of the prior art formulations.

The presently disclosed ready-to-use Sincalide formulation are adapted for administration by injection. These Sincalide formulations are characterized by improved stability and ease of commercial manufacturing.

In one or more embodiments, the Sincalide formulations include an effective amount of Sincalide, one or more stabilizers/chelator, and a buffer. In other embodiments, kits and methods for preparing improved Sincalide formulations, as well as methods for treating, preventing, and diagnosing gall bladder-related disorders using Sincalide formulations, are disclosed. The formulations may be employed to stimulate gallbladder contraction, as may be assessed by various methods of diagnostic imaging, or to facilitate obtaining, by duodenal aspiration, a sample of concentrated bile for analysis of cholesterol, bile salts, phospholipids, and crystals; stimulate pancreatic secretion (especially in conjunction with secretin) prior to obtaining a duodenal aspirate for analysis of enzyme activity, composition, and cytology; and accelerate the transit of a barium meal through the small bowel, thereby decreasing the time and extent of radiation associated with fluoroscopy and x-ray examination of the intestinal tract.

In accordance with one or more embodiment, a pharmaceutical composition includes a ready-to-use, stable, pharmaceutically acceptable formulation of Sincalide including a physiologically effective amount of Sincalide, and at least one pharmaceutically acceptable excipient selected from a stabilizer, a chelator, a buffering agent and a tonicity adjuster wherein the composition does not include a surfactant.

The pharmaceutically acceptable excipient may be a multi-functional excipient which performs the function of a stabilizer and chelator, tonicity adjuster, and a buffer.

Suitable buffers include, but are not limited to, phosphate, citrate, sulfosalicylate, borate, acetate and amino acid buffers. Phosphate buffers, such as but not limited to dibasic potassium phosphate, are preferred.

Various embodiments of the invention preferably include the stabilizerichelator EDTA.

In one or more embodiments the pharmaceutical composition includes EDTA in the amount of 0.01-0.1% w/v EDTA. In some embodiment the amount of EDTA is 1 mg/5 mL,

In one or more embodiments the pharmaceutical composition includes potassium phosphate (dibasic) in the amount of 1.1 to 1.8 mg/mL. In some embodiments the amount of potassium phosphate (dibasic) is 1.8 μg/mL.

In some embodiments, ready-to-use formulations include 0.0008 to 0.0012 mg/mt Sincalide. In particular embodiments, the ready-to-use formulation includes about 0.001 mg/mL Sincalide, about 0.01-0.1% w/v EDTA (disodium edetate) and about 1.8 mg/mL potassium phosphate (dibasic).

In one or more embodiments the pharmaceutical composition includes methyl paraben in the amount of 0.2 mg/mL.

In one or more embodiments the pharmaceutical composition includes propyl paraben in the amount of 0.1 mg/mL.

In one or more embodiments the pharmaceutical composition includes sodium chloride in the amount of 1.8 mg/mL.

In one or more embodiments the pH of the composition is in the range of 6.0 to 8.0. In some embodiments the pH is 7.5.

In one or more embodiments the osmolality of the composition is in the range of 180 to 320 mOsm/kg.

In some embodiments, a finished pharmaceutical product may, for example, include various components of the formulation as a mixture in liquid form, along with a container (e.g., a vial or a syringe) to hold the liquid mixture and a physiologically acceptable vehicle. In or more embodiments a ready-to-use sterile injectable pharmaceutical product includes an aqueous solution of Sincalide and at least one excipient selected from a stabilizer, a chelator, a tonicity adjuster, and a buffer, and a sterile container. In some embodiments the sterile container is a vial or a syringe which holds the ready-to-use aqueous solution.

The formulations disclosed herein have improved stability and potency compared to previous Sincalide formulations, and are useful as diagnostic aids for imaging the hepatobiliary system of a patient. When used as a diagnostic aid, the Sincalide formulations may, for example, be co-administered with a radiopharmaceutical agent having rapid hepatic uptake, such as ^{99 m}Tc-mebrofenin, or similar hepatobiliary imaging agents, to assist in the diagnosis of gall-bladder diseases and related disorders. Additionally, the formulations may be administered before and/or after diagnostic imaging (including for example, magnetic resonance imaging, scintigraphic imaging, ultrasound imaging, etc.).

Accordingly, methods are disclosed for imaging the hepatobiliary system of a subject, the method including: (a) administering a hepatobiliary imaging agent to the subject; (b) before or after step (a), administering to the subject a Sincalide formulation including a physiologically effective amount of Sincalide, and at least one pharmaceutically acceptable excipient selected from a stabilizer, a chelator, a buffering agent and a tonicity adjuster wherein the composition does not include a surfactant; and (c) detecting the imaging agent in the subject with a detection device.

In another embodiment a method is disclosed for imaging the hepatobiliary system of a subject, the method including: (a) administering to a subject a Sincalide formulation including a physiologically effective amount of Sincalide and at least one pharmaceutically acceptable excipient selected from a stabilizer, a chelator, a buffering agent and a tonicity adjuster, and (b) scanning the subject using a diagnostic imaging modality.

The ready-to-use Sincalide formulations disclosed herein may also be administered to patients receiving total parenteral nutrition (TPN), in order to treat and/or prevent TPN-related disorders. In one or more embodiments a method is disclosed for treating or preventing a medical condition associated with total parental nutrition (TPN), the method including administering to a patient receiving TPN an effective amount of a Sincalide formulation, the formulation including Sincalide and one or more excipients which perform the function of a stabilizer, a chelator, a tonicity adjuster, and a buffer.

Also disclosed herein is a method of making a pharmaceutically acceptable, ready-to-use stabilized formulation of Sincalide, the method including the step of combining a physiologically effective amount of Sincalide, and one or more excipients which independently or in combination perform the function of a stabilizer, a chelator, tonicity adjuster and a buffer.

Other features and advantages of the invention will be apparent from the following detailed description thereof and from the claims.

### DETAILED DESCRIPTION OF THE INVENTION

The present invention now will be described more fully hereinafter with reference to the accompanying examples and experiments, in which illustrative embodiments of the invention are shown. This invention may, however, be embodied in many different forms and should not be construed as limited to the embodiments set forth herein; rather these embodiments are provided so that this disclosure be thorough and complete, and will fully convey the scope of the invention to those skilled in the art.

The terminology used herein is for the purpose of describing particular embodiments only and is not intended to be limiting of the invention. As used herein, the singular forms “a”, “an” and “the” are intended to include the plural forms as well, unless the context clearly indicates otherwise. Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

It has surprisingly been found that the potency and stability of Sincalide formulations can be significantly enhanced through the careful selection of excipients that provide certain desired function. Accordingly, the embodiments disclosed herein provide novel ready-to-use Sincalide formulations having improved stability and/or potency over previous formulations and eliminate the need for freeze drying, and reconstitution with water for injection at the time of use.

As used herein, the term “Sincalide” includes the synthetically-prepared C-terminal octa-peptide of cholecystokinin (CCK-8), with the amino acid sequence: Asp-Tyr(S03H)-Met-Gly-Trp-Met-Asp-Phe-NH2, as well as derivatives thereof which have been modified to improve stability, potency, pharmacokinetics, etc., but retain the biological activity of the original octapeptide. For example, peptides in which the stabilizer/chelator chosen to replace other ingredients without significantly affecting biological activity are included within “Sincalide” as the term is used herein. Similarly, the term “Sincalide” encompasses not only monomeric, but multimeric forms of the peptide, as well as physiologically active degradants or portions of the peptide and its derivatives.

Table 1 below shows the concentration ranges for various excipients that were investigated. In general, the range studies were based on a fill of bulk solution per vial. The final Sincalide formulation results in an isotonic solution. The concentration ranges of the various ingredients provided in Table 1 can be adjusted upward or downward, if necessary in conjunction with: increasing or decreasing the fill volume per vial, obtaining the desired pH, and the desirability of achieving tonicity in the final solution. For example, as indicated above, the concentrations provided in Table 1 were developed to provide an isotonic solution; however, one skilled in the art would recognize that a broader range of concentrations could be used if an isotonic solution was not required.

TABLE 1

| Item | | Lot # SSI/739/879/009 |
|---|---|---|
| no. | Formulation Ingredient | QTY (mg)/vial (5 mL) |
| 1 | Sincalide | 5 |
| 2 | EDTA DISODIUM | 1 |
| 3 | Methyl Paraben | 1 |
| 4 | Propyl Paraben | 0.5 |
| 5 | Benzyl Alcohol | NA |
| 5 | WFI | q.s. to 5 mL |

Table 2 shows exemplary ranges for excipients in the bulk, ready-to-use solution filled into vials or syringes. All concentrations shown for the bulk solution are based on a 5 mL fill volume. The ingredient quantities are matched to result in a pH slightly below neutral and result in an isotonic solution in a ready-to-use vial as indicated by an osmolality in the range of 180 to 320.

TABLE 2

| Item | Formulation | | | QTY (mg)/ |
|---|---|---|---|---|
| no. | Ingredient | QTY (mg)/mL | QTY (mg)/mL | mL |
| 1 | Sincalide | 1 | 1 | 1 |
| 2 | EDTA Disodium | 0.2 | 0.2 | 1 |
| 3 | Methyl Paraben | 0.2 | 0.2 | 0.5 |
| 4 | Propyl Paraben | 0.1 | 0.1 | N/A |
| 5 | WFI | q.s. to 1 mL | q.s. to 1 mL | q.s. to 1 mL |

### Chelators

Excipient impurities and/or stopper extractables can introduce trace metals into pharmaceutical formulations. Embodiments of the Sincalide formulations disclosed herein contain stabilizerichelator to inhibit the oxidation of the two methionine residues present in Sincalide (Met 3 and Met 6). Preferred chelators include edetic acid (EDTA) and derivatives thereof, including salts. It has been found the amounts of the degradants Sincalide Met 3 and Sincalide Met 6 mono sulfoxides, increase in the presence of certain metals and in the absence of EDTA, while the presence of EDTA has an inhibitory effect on the formation of these mono sulfoxides. In particular, copper and manganese, in the absence of EDTA, have the greatest oxidative effect on the methionine residues of Sincalide.

In a preferred embodiment, the Sincalide formulations contain between 0.1 and 3.0 mg of EDTA per mL after reconstitution. In a particularly preferred embodiment, Sincalide formulations of the invention contain 0.2 mg EDTA per mL.

### Buffering Agents

Buffering agents are employed to stabilize the pH of Sincalide formulations of the invention, and consequently, reduce the risk of chemical stability at extreme pH values. Buffering agents useful in the preparation of formulation of the invention include, but are not limited to, phosphoric acid, monobasic or dibasic sodium phosphate, monobasic or dibasic potassium phosphate, citric acid, sodium citrate, sulfosalicylate, acetic acid, potassium acetate, sodium acetate, methyl boronic acid, disodium succinate hexahydrate, amino acids, including amino acid salts, lactic acid, sodium lactate, maleic acid, maleate, potassium chloride, benzoic acid, sodium benzoate, carbonic acid, sodium carbonate, sodium bicarbonate, boric acid, sodium borate, sodium chloride, succinic acid, sodium succinate, tartaric acid, sodium tartrate, tris-(hydroxy methyl) amino methane, and biological buffers.

Phosphate is an effective and desirable buffering agent due to its lack of interaction with Sincalide and an ideal buffering capacity in the physiological pH range. Dibasic potassium phosphate is a particularly preferred buffer in Sincalide formulations of the invention. As described in Example 1 below, a Sincalide formulation of the invention proved to be stable over a pH range of 5.5-9.1. Within the pH range of 5.5-8.5, no distinct pH-dependent related trends in initial Sincalide recovery were observed with a Sincalide formulation of the invention. Preferably, a Sincalide ready-to-use formulation of the invention has a pH from 6.0 to 8.0.

### Stabilizers

The octapeptide, Sincalide, contains one tryptophan and two methionine residues. Methionine has been identified as one of the most easily oxidizable amino acids, which degrades to its corresponding sulfoxide and, under more strenuous oxidation conditions, its sulfone. The mechanisms of oxidation appear to be highly dependent on the reactive oxygen species under consideration: peroxide, peroxyl radicals, singlet oxygen, and hydroxyl radical have all been shown to oxidize methionine residues to sulfoxides and other products. Therefore, based on the potential for oxidation of this peptide, it was necessary to identify functional excipients for peptide stabilization

### Antioxidants/Reducing Agents

In a desirable embodiment of the invention, the Sincalide formulation contains an antioxidant or reducing agent as a stabilizer. A wide variety of antioxidants or reducing agents can be used as stabilizers, including but not limited to, acetylcysteine, cysteine, ascorbic acid, benzyl alcohol, citric acid, propyl gallate, methylparaben, sulfoxylate, propyl paraben, edetic acid or ethylene diamine tetra acetic acid (EDTA), disodium EDTA dihydrate, glutathione, potassium metabisulfite, sodium formaldehyde sulfoxylate, sodium sulfite, sodium succinate, sodium metabisulfite, stannous chloride, thioacetic acid, thiodiglycerol, thioethanolamine, thioglycolic acid, 2-aminoethanethiol (cysteamine), butylated hydroxyanisole (BHT), and sodium sulfate and derivatives thereof, including salts and sulfurous acid salts. Disodium EDTA dihydrate is a preferred antioxidant stabilizer.

### Tonicity Adjusters

Due to the small amount of Sincalide present in the ready-to-use formulations of the invention, tonicity adjusters are useful to provide structure and support for the active ingredient, Sincalide, as well as to provide tonicity. Of these, sodium chloride is a desirable tonicity adjuster for use with the invention.

### Other Excipients

Other excipients, which may optionally be used in embodiments disclosed herein include preservatives (e.g., benzalkonium chloride), osmolality adjusters (e.g., sodium chloride, dextrose), tonicity adjusters (e.g. sodium chloride), complexing agents etc. A listing of various excipients that can be used in Sincalide ready-to-use formulations for parenteral administration can be verified by references listed in the reference section.

Embodiments of the present invention are illustrated by the following examples, which are in no way intended to be limiting of the invention.

### EXAMPLE 1—Effect of Buffering Agent and Formulation pH: on Ready-to-Use Sincalide Formulations

Experiments were conducted to determine the effect of pH on the chemical stability of Sincalide. Chemical instability, or degradation, may be caused by, for example, oxidation, reduction, de-amidation, hydrolysis, imide formation, racemization, isomerization, and/or β-elimination. To examine the effect of pH on Sincalide in phosphate buffer solution, solutions of Sincalide (˜1.7, μg/mL) were prepared in 35 mM phosphate buffer and pH-adjusted with either dilute HCl or NaOH for final pH values ranging from 3.0-9.1. Using reverse-phase HPLC (RP-HPLC) with gradient elution and UV detection at 215 nm, Sincalide stability in solution was assessed by measuring the recovery of Sincalide at 0, 6, and 24 hours after pH adjustment.

Based on the results, phosphate was selected as the buffering agent of choice due to a lack of interaction with Sincalide and an ideal buffering capacity in the physiological pH range. Subsequently, experiments using phosphate in the formulation shown in Table 3 over the stable pH range established above were performed. Briefly, solutions of Sincalide containing the following components (in the concentrations indicated in Table 3) were prepared: Sincalide, EDTA, methyl paraben, propyl paraben, sodium chloride and dibasic potassium phosphate.

### Formulation Details

The formulation of the present invention may include one or more vials or syringes containing the ready-to-use sterile formulation of a predetermined amount of Sincalide, tonicity adjuster, one or more stabilizers/chelator, and a buffer. The one or more vials that contain all or part of the formulation can independently be in the form of a vessel containing sterile solution. Buffering agents useful in the preparation of formulation of the invention are discussed herein and include, for example phosphate, citrate, sulfosalicylate, and acetate, and amino acids (including amino acid salts). Dibasic potassium phosphate is a preferred buffer in Sincalide formulations of the invention.

As discussed, a component in a ready-to-use formulation according to the present disclosure can also serve more than one function. For example, an excipient which serves as a stabilizer may also serve as the chelator and a tonicity adjuster. In addition, in some embodiments, the excipients are added in dry powder form, or all in liquid form while in other embodiments, some of the excipients are in dry form and others are in a fluid form. A particularly preferred ready-to-use vial or syringe of the invention contains about 0.005 mg Sincalide, about 2 mg EDTA, about 0.04 mg sodium metabisulfite, about 9 mg dibasic potassium phosphate.

TABLE 3
Components of a Sincalide Formulation for Example 1

| | Concentration | |
|---|---|---|
| Component | (mg/vial) | Function |
| Sincalide | 0.0050 | Active |
| EDTA | 2.0 | Chelator/Stabilizer |
| Methyl Paraben | 1.0 | Preservative |
| Propyl Paraben | 0.5 | Preservative |
| Sodium Metabisulfite | 0.040 | Stabilizer |
| Dibasic Potassium Phosphate | 9.0 | Buffer |

Solutions were pH-adjusted from 5.5-8.5 with dilute HCl or NaOH. and were evaluated for stability by measuring the Sincalide recoveries at 0, 4, and 8 hours after pH adjustment, using RP-HIPLC with gradient elution and UV detection at 215 nm, as described above. An 8-hour study on the stability of Sincalide in the above formulation over the pH range of 5.5-8.5 was conducted.

A 24-hour study on the stability of Sincalide in phosphate buffer over the pH range of 3.0-9.1 was conducted. By measure of the percentage recovery, Sincalide was stable in 35 mM phosphate buffer solution at pH values ranging from 5.0-9.1 over a 24-hour period. At pH values <5.0, Sincalide degradation was evident even at the initial time point.

### Therapeutic/Diagnostic Uses and Methods

Sincalide is a synthetic analog of the endogenously produced hormone cholecystokinin (CCK-8). CCK-8 acts on receptors within the gallbladder wall causing it to contract, cleaning out any remaining sludge or bile that may have accumulated within the gallbladder. CCK-8 increases bile flow and small and large bowel motility, causes the pyloric sphincter to contract and increases pancreatic enzyme secretion. CCK-8 also causes delayed biliary to bowel transit. Sincalide has a more rapid physiologic effect on the gallbladder in terms of contraction and relaxation than the endogenous hormone (CCK-8) produced by the body, making Sincalide formulations useful as diagnostic aids for hepatobiliary imaging, when administered alone or in conjunction with a hepatobiliary imaging agent. For example, Sincalide may be administered before and/or after diagnostic imaging (for example, magnetic resonance imaging, scintigraphy imaging, ultrasound imaging, etc.) to improve visualization and/or diagnosis of various disease states.

In one embodiment, hepatobiliary imaging can be performed using, for example, hepatobiliary scintigraphy, an instrumental imaging tool used in the diagnosis and evaluation of hepatobiliary disease. Detection of diseases, such as acute and chronic cholecystitis, biliary obstruction, bile leaks, and other forms of hepatobiliary disease, help the physician to better determine the appropriate course of treatment and management of the patient suffering from a suspected hepatobiliary pathology.

As explained below, the indications for use of Sincalide in conjunction with hepatobiliary imaging include pretreatment of patients who have not eaten for more than 20 to 24 hours prior to imaging (in order to empty the gallbladder (GB) of non-radiolabeled bile) and use in the analysis of gallbladder motor function, including the determination of GBEF (gallbladder ejection fraction).

It is important to properly prepare the patient prior to hepatobiliary imaging in order to achieve high quality imaging and reduce the number of false positive and negative results. Preferably, patients should have nothing to eat for 4 to 1.2 hours prior to hepatobiliary imaging. Prolonged fasting, however, may result in false positive test results (i.e. failure to visualize the gallbladder). If a patient has not eaten for more than 24 hours, the patient is preferably pretreated with Sincalide by administration of the Sincalide formulation described herein prior to imaging. Typically, the gallbladder contracts within 15 minutes after Sincalide injection and the hepatobiliary imaging agent (e.g., radiotracer) is injected 30 minutes later. The gallbladder is then emptied and is better able to take up and accumulate imaging agent (e,g., radiotracer), which helps to reduce the number of false positive studies.

The preferred radiopharmaceuticals used for hepato-biliary imaging include_{;} but are not limited to, Tc 99m IDA (iminodiacetic acid) analogs, such as Tc-99m mebrofenin (CHOLETEC®), Tc-99m disofenin (DISIDA), and Tc-99m lidofenin (see, U.S. Pat. No. 4,418,208). Tc-99m mebrofenin is a preferred hepatobiliary imaging agent. Methods for co-administration of Tc 99m IDA (Iminodiacetic acid) analogs with CCK and Sincalide are known in the art and described in, for example, Ziessman H A, et al,, Cholecystokinin cholescintigraphy: victim of its own success? 1 Nucl. Med. 1999, 40:2038-2042; Krishnamurthy S., et al., Gallbladder ejection fraction.

### Administration of Sincalide Formulations via IV or INT Injections

For IV administration the dose can be administered as a bolus or slow injection over time optionally with the aid of an infusion pump. The dose for IV administration is typically 0.005 to 0.04 μg/kg (bolus injection) or 0.005 μg/kg in a series of 4-three minute injections. A dose of 0.02-0.04 μg/kg IV over 2-3 minutes, but up to 1 hour is described in the art. Injection rates of 0.58 μg/kg/hour can also be employed with the use of an infusion pump. Other regimens starting at 10 ng/kg/hr. and increasing to 160 ng/kg/hr. are also known in the art. Bolus injection is not recommended in every case, but injection of 0.02 to 0.04 μg/kg over 2-3 minutes even up to 15 min. can be used to avoid spasm of the cystic duct or GB.

Doses for IM administration are generally higher and range from 0.1 to 0.4 μg/kg. In one embodiment the 0.4 ₗag/kg IM dose is generally preferred resulting in the greatest GB response with the fewest side effects. Further details on administration are provided in, for example, Mesgarzadeh M., et al,, Filling, post cholecystokinin emptying and refilling of normal gallbladder. J. Nucl. Med. 1983, 24:666-671; Ziessmann H A; et al., Cholecystokinin cholescintigraphy: victim of its own success? 1 Nucl, Med. 1999, 40:2038-2042.

The Sincalide formulations of the invention are also useful for treating patients receiving total parenteral nutrition (TPN). TPN induces biliary sludge, the development of cholestasis, and the formation of gall stones and other gallbladder related complications. Indeed, TPN associated cholestasis (TPN-AC) can be a fatal in some instances. The clinical implications of TPN-AC include increased rates of sepsis, cirrhosis, declined lymphocyte function, obstructive jaundice, liver failure, and increased mortality. Although the mechanisms by which these disorders develop have not been definitely established, biliary stasis, the reduction in gall-bladder emptying, bile flow, and bile acid secretion that treat high bilinthin levels the dose is 0.02 μg/kg IV or MI twice or 3 times daily with doses increasing up to 0.32 μg/kg. CCK induces not only GB contraction but also increases intra-hepatic bile flow. Information on the treatment of TPN-patients is provided in, for example, Sitzmann, J. V., et al., Cholecystokinin prevents parenteral nutrition induced biliary sludge in humans, Surg. Gynecol. Obstet. Vol. 170:25-31, 1990; Moss R L, et al., New approaches to understanding the etiology and treatment of total parenteral nutrition-associated cholestasis, Surg. Gynecol. Obstet. Vol. 8:140-147, 1999; Teitelbaum D H., et al., Treatment of parenteral nutrition-associated cholestasis with cholecystokinin-octapeptide. J. Pediatr. Surg. 30:1082, 1995; Teitelbaum D H. Parenteral nutrition-associated cholestasis, Current Opinion in Pediatrics 1997. 9:270-275; Teitelbaum D H., et al., Parenteral nutrition-associated cholestasis. Seminars in Pediatric Surgery, Vol. 10, pp. 72-80.

Although the formulations, schemes and methods of the present disclosure have been described with reference to exemplary embodiments thereof, the present disclosure is not limited thereby. Indeed, the exemplary embodiments are implementations of the disclosed methods are provided for illustrative and non-limitative purposes. Changes, modifications, enhancements and/or refinements to the disclosed methods may be made without departing from the spirit or scope of the present disclosure. Accordingly, such changes, modifications, enhancements and/or refinements are encompassed within the scope of the present invention. All publications, patent applications, patents, figures and other references mentioned herein are expressly incorporated by reference in their entirety.

### REFERENCES

The Handbook of Pharmaceutical Additives, Second Edition, edited by Michael & Irene Ash. Remington's Pharmaceutical Sciences, (22^{nd} Edition), edited by Loyd V. Allen, 2013.
PDA Journal of Pharmaceutical Science and Technology, 53(6):324 (1999).
Strickly, Robert G., Parenteral Formulations of Small Molecules Therapeutics Marketed in the United States (1999)-Part IT. PDA Journal of Pharmaceutical Science and Technology, 54(1):69 (2000).
PDA Journal of Pharmaceutical Science and Technology, 54(2): 154 (2000).
Nem, Sandeep, et al., Excipients and Their Use in Injectable Products, PDA Journal of Pharmaceutical Science and Technology, 51(4): 166 (1997).
Wang, Y. J., et al., Parenteral Formulations of Proteins and Peptides; Stability and Stabilizers (Technical Report No. 10), Journal of Parenteral Science and Technology, Vol. 42 (2S), Supplement 1988.
Shah, D et al., the Effects of Various Excipients on the Unfolding of Basic Fibroblast Growth Factor, PDA Journal of Pharmaceutical Science &. Technology, 52(5):238 (1998).
Powell, M. F., et al., Compendium of Excipients for Parenteral Formulations, PDA Journal of Pharmaceutical Science & Technology, 52(5):238 (1998).
Inactive Ingredient Guide, Div. Of Drug Information Resources, FDA, CDER, January 1996.
Handbook of Injectable Drugs, Edition 8, Am. Soc. Hospital Pharmacists, 1994.

1. (canceled)

2. A stable, ready-to-use composition comprising a physiologically effective amount of Sincalide, and at least one pharmaceutically acceptable excipient.

3. The stable, ready-to-use composition according to claim 1, wherein the composition comprises an aqueous solution of Sincalide, and the composition is suitable for administration by injection.

4. The stable, ready-to-use composition according to claim 2, wherein the at least one pharmaceutically acceptable excipient is selected from a stabilizer, a chelator, a tonicity adjuster, and a buffer.

5. The stable, ready-to-use composition according to claim 3, wherein the stabilizer is an antioxidant or a reducing agent.

6. The stable, ready-to-use composition according to claim 3, wherein the chelator is EDTA or a derivative thereof. (New) The stable, ready-to-use composition according to claim 3, wherein the tonicity adjuster is sodium chloride.

8. The stable, ready-to-use composition according to claim 3, wherein the buffer is selected from phosphoric acid, monobasic or dibasic sodium phosphate, monobasic or dibasic potassium phosphate, citric acid, sodium citrate, sulfosalicylate, acetic acid, potassium acetate, sodium acetate, methyl boronic acid, disodium succinate hexahydrate, amino acids, amino acid salts, lactic acid, sodium lactate, maleic acid, maleate, potassium chloride, benzoic acid, sodium benzoate, carbonic acid, sodium carbonate, sodium bicarbonate, boric acid, sodium borate, sodium chloride, succinic acid, sodium succinate, tartaric acid, sodium tartrate, tris-(hydroxy methyl amino methane, and biological buffers.

9. The stable, ready-to-use composition of claim 2, wherein the composition is contained in a vial or syringe.

10. The stable, ready-to-use composition of claim 2, wherein the composition has a pH from 5.5 to 9.1.

11. The stable, ready-to-use composition of claim 3, wherein the stabilizer is disodium EDTA dihydrate.

12. A method for treating, preventing, or diagnosing gall bladder-related disorders, comprising administering to a subject in need thereof; the injectable composition as claimed in claim 1 in an amount effective to treat, prevent, or diagnose gall-bladder-related disorders.
